(19) United States
(12) Patent Application Publication
(10) Pub. No.: US 2016/0038658 A1
(43) Pub. Date: Feb. 11, 2016

(54) FLUID COLLECTOR UNIT OF WOUND DRAINAGE THERAPY SYSTEM AND COLLECTION CONTAINER THEREOF

(71) Applicant: APEX MEDICAL CORP., New Taipei City (TW)

(72) Inventors: Chih-Tsan CHIEN, New Taipei City (TW); Chiu-Yu YEH, New Taipei City (TW); Sih-Jhe YANG, New Taipei City (TW); Yu-Hao CHEN, New Taipei City (TW)

(21) Appl. No.: 14/919,032

(22) Filed: Oct. 21, 2015

Related U.S. Application Data
(63) Continuation-in-part of application No. 13/685,027, filed on Nov. 26, 2012, now Pat. No. 9,199,010.

(51) Int. Cl. A61M 1/00

(52) U.S. Cl. CPC A61M 1/0011; A61M 1/0023; A61M 1/0086; A61M 2205/0205

(57) ABSTRACT

A fluid collector unit of a wound drainage therapy system includes a multiple-pipe integration module and a collection bag. The multiple-pipe integration module includes first, second and third connection ports. The collection bag includes a negative-pressure buffer zone and two fluid collection zones that communicate with each other. The negative-pressure buffer zone is located between the two fluid collection zones and isolated from the two fluid collection zones. The two fluid collection zones are connected with the first connection port of the multiple-pipe integration module. The negative-pressure buffer zone is connected with the second and third connection ports of the multiple-pipe integration module, and the negative-pressure buffer zone has a fluid input port.

### CROSS-REFERENCE TO RELATED APPLICATIONS

This application is a Continuation-In-Part (CIP) of U.S. patent application Ser. No. 13/685,027 filed Nov. 26, 2012, the contents of which are incorporated herein by reference in their entirety.

### FIELD OF THE INVENTION

The present invention relates to a fluid collector unit of a wound caring system. More particularly, the present invention relates to a fluid collector unit of a negative pressure wound drainage therapy system.

### BACKGROUND OF THE INVENTION

Negative pressure wound therapy employs a vacuum pump to provide a negative pressure environment for a wound so as to extract wound pus and infected material, to attract the healthy tissue fluid to maintain a moist healing environment, and to promote the surrounding blood microcirculation, thereby accelerating wound healing effect.

In order to cope with the negative pressure wound therapy, there are a lot of negative pressure wound care devices came into being. However, the overall volume of a conventional negative pressure wound care device is too large such that it is not conducive to the patient carries and thus limited to the patients stayed in hospital. In addition, the conventional negative pressure wound care device is equipped with several pipes such that it is prone to the disadvantage of pipe entanglement that also causes the patients inconvenient. For the forgoing reasons, there is a need for further improving the negative pressure wound care device, so that the negative pressure wound therapy can benefit more patients.

### SUMMARY OF THE INVENTION

It is therefore an objective of the present invention to provide an improved fluid collector unit of a wound drainage therapy system.

In accordance with the foregoing and other objectives of the present invention, a fluid collector unit of a wound drainage therapy system includes a multiple-pipe integration module and a collection bag. The multiple-pipe integration module includes first, second and third connection ports. The collection bag includes a negative-pressure buffer zone and two fluid collection zones that communicate with each other. The negative-pressure buffer zone is located between the two fluid collection zones and isolated from the two fluid collection zones. The two fluid collection zones are connected with the first connection port of the multiple-pipe integration module. The negative-pressure buffer zone is connected with the second and third connection ports of the multiple-pipe integration module, and the negative-pressure buffer zone has a fluid input port.

According to another embodiment disclosed herein, the negative-pressure buffer zone has a negative-pressure detection isolation section that is connected with the third connection port of the multiple-pipe integration module.

According to another embodiment disclosed herein, the negative-pressure buffer zone and the two fluid collection zones all have polyvinyl alcohol sheets inside thereof.

According to another embodiment disclosed herein, the multiple-pipe integration module further comprises fourth, fifth, sixth and seventh connection ports, and the first, fourth and seventh connection ports communicate with one another. The second and fifth connection ports communicate with each other, the third and sixth connection ports communicate with each other, and the seventh connection port contains a backwater gate inside to stop flows reversed from the first or fourth connection ports.

According to another embodiment disclosed herein, the first, second, third, fourth, fifth, sixth and seventh connection ports are located on a common flat surface of the multiple-pipe integration module.

According to another embodiment disclosed herein, each of the two fluid collection zones has a ventilation hole and a waterproof ventilation sheet that is attached to the ventilation hole.

In accordance with the foregoing and other objectives of the present invention, a fluid collector unit of a wound drainage therapy system includes a multiple-pipe integration module and a collection container. The multiple-pipe integration module includes first, second, third and fourth connection ports. The collection container includes a negative-pressure buffer zone, a positive-pressure detection zone and a fluid collection zone. The negative-pressure buffer zone is isolated from the positive-pressure detection zone and the fluid collection zone. The fluid collection zone is connected with the first connection port of the multiple-pipe integration module. The positive-pressure detection zone is connected with the fourth connection port of the multiple-pipe integration module and communicates with the fluid collection zone. The negative-pressure buffer zone is connected with the second and third connection ports of the multiple-pipe integration module, and the negative-pressure buffer zone has a fluid input port.

According to another embodiment disclosed herein, the negative-pressure buffer zone has a negative-pressure detection isolation section that is connected with the third connection port of the multiple-pipe integration module; and the positive-pressure detection zone has a positive-pressure detection isolation section that is connected with the fourth connection port of the multiple-pipe integration module.

According to another embodiment disclosed herein, the negative-pressure buffer zone has a polyvinyl alcohol sheet inside thereof, and the fluid collection zone has superabsorbent polymer materials inside thereof.

According to another embodiment disclosed herein, the multiple-pipe integration module further comprises fifth, sixth, seventh and eighth connection ports. The first and fifth connection ports communicate with each other, the second and sixth connection ports communicate with each other, the third and seventh connection ports communicate with each other, and the fourth and eighth connection ports communicate with each other.

According to another embodiment disclosed herein, the first, second, third, fourth, fifth, sixth, seventh and eighth connection ports are located on a common flat surface of the multiple-pipe integration module.

According to another embodiment disclosed herein, the fluid collection zone has at least one ventilation hole and at least one waterproof ventilation sheet that is attached to the at least one ventilation hole.

According to another embodiment disclosed herein, the fluid collector unit further comprises a first connection pipe and a second connection pipe. The first connection pipe connects the fluid collection zone with the first connection port of the multiple-pipe integration module, and the second connection pipe connects the negative-pressure buffer zone with the second connection port of the multiple-pipe integration module. Each of the first connection pipe and the second connection pipe has a muffler element inside thereof.

In accordance with the foregoing and other objectives of the present invention, a collection container includes a negative-pressure portion and a positive-pressure portion. The negative-pressure portion includes a negative-pressure buffer zone, a fluid input port, a fluid output pipe and a first gas output pipe, the fluid output pipe, the first fluid input pipe and the first gas output pipe are communicated with the negative-pressure buffer zone respectively. The positive-pressure portion includes a positive-pressure detection zone, a fluid collection zone, a second fluid input pipe and a second gas output pipe, the negative-pressure buffer zone is isolated from the fluid collection zone and the positive-pressure detection zone; the second fluid input pipe is communicated with the fluid collection zone, the second gas output pipe is communicated with the positive-pressure detection zone, and the positive-pressure detection zone is communicated with the fluid collection zone.

According to another embodiment disclosed herein, the negative-pressure buffer zone has a negative-pressure detection isolation section that is connected with the first gas output port; and the positive-pressure detection zone has a positive-pressure detection isolation section that is connected with the second gas output port.

According to another embodiment disclosed herein, the negative-pressure buffer zone has a polyvinyl alcohol sheet inside thereof, and the fluid collection zone has superabsorbent polymer materials inside thereof.

According to another embodiment disclosed herein, the superabsorbent polymer materials can be polyvinyl alcohol sheets or sodium polyacrylate sheets.

According to another embodiment disclosed herein, the superabsorbent polymer materials have deodorant additives or antibacterial additives inside thereof.

According to another embodiment disclosed herein, the fluid collection zone has at least one ventilation hole and at least one waterproof ventilation sheet that is attached to the at least one ventilation hole.

According to another embodiment disclosed herein, each of the first fluid output pipe and the second fluid input pipe has a muffler element inside thereof.

Thus, the fluid collector unit of the wound drainage therapy system disclosed herein has modularized parts which can be easily assembled and disassembled with each other, the multiple-pipe integration module of which overcomes the piping entanglement problem, and the fluid collector unit is designed smaller to enable the wound drainage therapy system even more portable.

It is to be understood that both the foregoing general description and the following detailed description are by examples, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 illustrates an assembled view of a wound drainage therapy system according to an embodiment of this invention;

FIG. 2 illustrates an exploded view of the wound drainage therapy system in FIG. 1;

FIG. 3 illustrates an exploded view of a fluid collector unit in FIG. 1;

FIG. 4 illustrates an exploded view of a multiple-pipe integration module in FIG. 3;

FIG. 5 illustrates an enlarged view of a multiple-pipe integration module and a collection bag in FIG. 3;

FIG. 6 illustrates an enlarged view of a vacuum driving unit and an actuator in FIG. 2;

FIG. 7 illustrates a schematic view of the vacuum driving unit and the actuator connecting to the multiple-pipe integration module;

FIG. 8 illustrates an exploded view of the collection bag in FIG. 5;

FIG. 9 illustrates an exploded view of the vacuum driving unit in FIG. 6;

FIG. 10 illustrates an exploded view of the actuator in FIG. 6; and

FIG. 11 illustrates an exploded view of a wound seal unit in FIG. 2.

FIG. 12 illustrates an enlarged view of another example of a multiple-pipe integration module and a collection container of the fluid collector unit;

FIG. 13 illustrates an exploded view of the multiple-pipe integration module in FIG. 12;

FIG. 14 illustrates an exploded view of the collection container in FIG. 12.

### DETAILED DESCRIPTION OF THE INVENTION

Since various aspects and embodiments are merely exemplary and not limiting, after reading this specification, skilled artisans appreciate that other aspects and embodiments are possible without departing from the scope of the invention. Other features and benefits of any one or more of the embodiments will be apparent from the following detailed description and the claims.

Reference will now be made in detail to the present preferred embodiments of the invention, examples of which are illustrated in the accompanying drawings. Wherever possible, the same reference numbers are used in the drawings and the description to refer to the same or like parts.

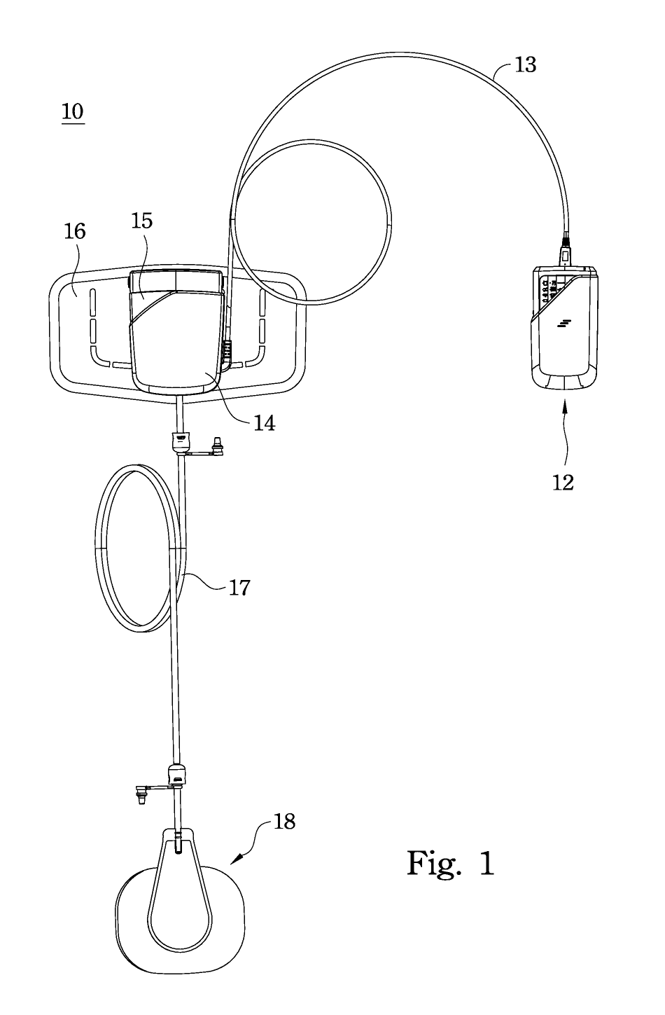

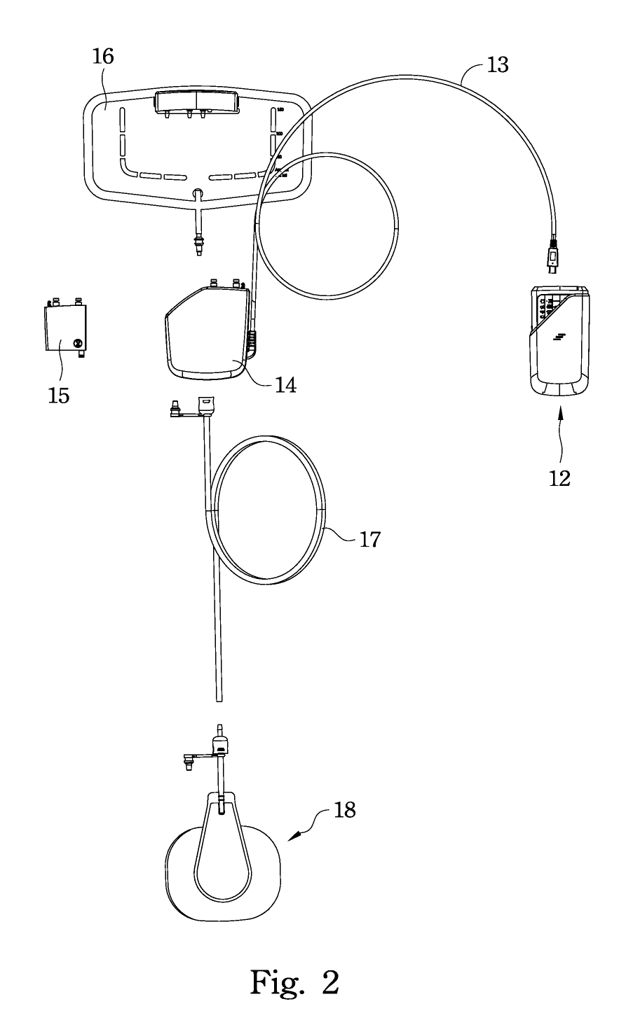

FIG. 1 illustrates an assembled view of a wound drainage therapy system according to an embodiment of this invention. FIG. 2 illustrates an exploded view of the wound drainage therapy system in FIG. 1. A modularized wound drainage therapy system 10 includes a control unit 12, an actuator 14, a vacuum driving unit 15, a fluid collector unit 16, a connection pipe 17 and a wound seal unit 18, wherein the fluid collector unit 16 and wound seal unit 18 are disposable parts. After the vacuum driving unit 15 is firstly used by a patient, the vacuum driving unit 15 would be infectious and thus should exclusively belong to the patient. The control unit 12 and actuator 14 are electronic parts of the wound drainage therapy system and can be repeatedly used in therapies for different patients. The control unit 12 is detachably connected with an electrical cable 13 of the actuator 14 such that the control unit 12 is able to control the operating of the actuator 14. The modularized wound drainage therapy system 10 not only make the fluid collector unit 16 smaller but also enable the electronic parts of high costs, e.g., the control unit 12 or actuator 14, to be repeatedly used in therapies for different patients, thereby reducing the using costs for the patients and preventing cross-infection among the patients.

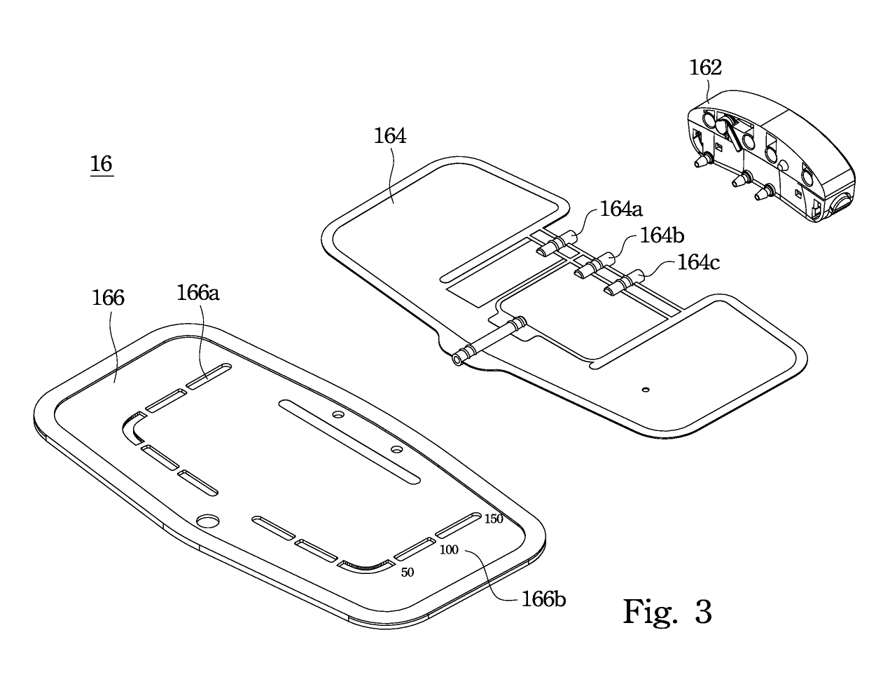

FIG. 3 illustrates an exploded view of a fluid collector unit in FIG. 1. The fluid collector unit 16 includes a multiple-pipe integration module 162, a collection container (e.g., a collection bag 164 or other likes) and an outer coat 166. The multiple-pipe integration module 162 is a single body for integrating all tubes connected to the collection bag 164 such that a wound drainage therapy system can be conveniently used and not bothered by piping entanglement. The collection bag 164 has three connection pipes (164 a, 164 b, 164 c) that are connected to the connection ports of the multiple-pipe integration module 162 respectively. The outer coat 166 is used to enclose the collection bag 164 and has several openings 166 a allowing a patient to check the fluid collection status of the collection bag via visual contacts. In addition, volume scales 166 b are labeled around the opening 166 a to assist estimating the fluid volume.

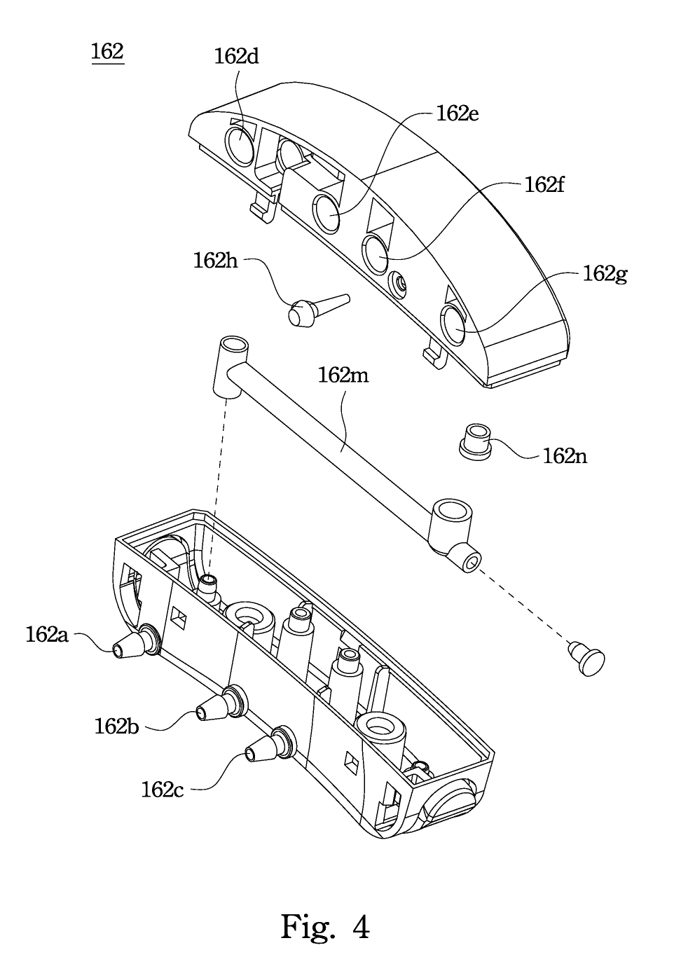

FIG. 4 illustrates an exploded view of a multiple-pipe integration module in FIG. 3. The multiple-pipe integration module 162 includes an upper half housing and a lower half housing to assemble a complete one. The multiple-pipe integration module 162 includes a first row connection port group and a second row connection port group. The first row connection port group includes first, second and third connection ports (162 a, 162 b, 162 c), and the second row connection port group includes fourth, fifth, sixth and seventh connection ports (162 d, 162 e, 162 f, 162 g). The first row connection port group and second row connection port group have the following connection relationship. The first, fourth and seventh connection ports (162 a, 162 d, 162 g) communicate with one another by means of a three-way connection pipe 162 m, the second and fifth connection ports (162 b, 162 e) communicate with each other, and the third and sixth connection ports (162 c, 162 f) communicate with each other. In addition, the seventh connection port 162 g contains a backwater gate 162 n inside thereof to stop flows reversed from the first or fourth connection ports (162 a, 162 d).

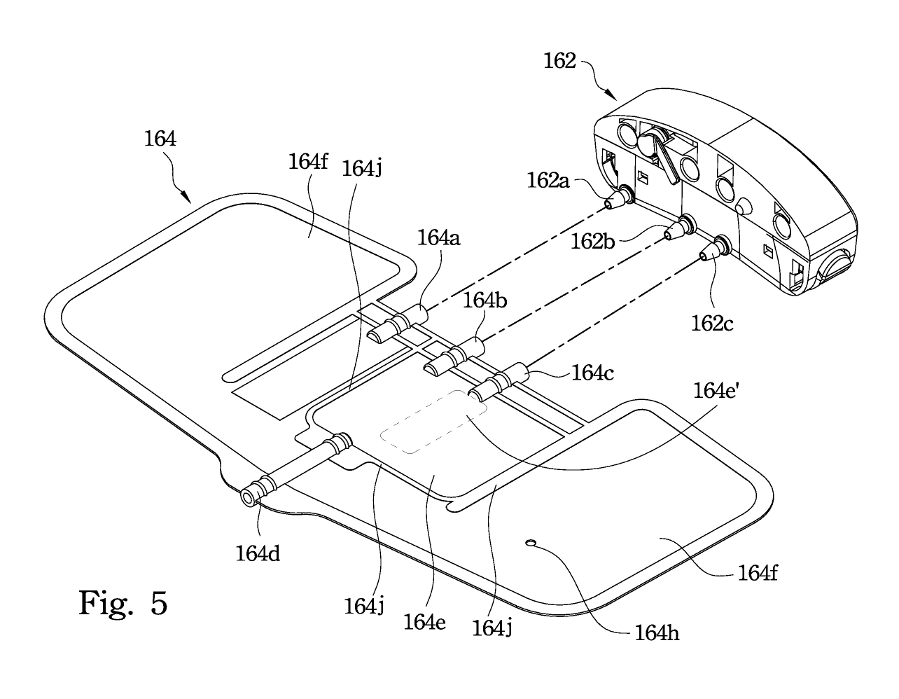

FIG. 5 illustrates an enlarged view of a multiple-pipe integration module and a collection bag in FIG. 3. The multiple-pipe integration module 162 includes first, second and third connection ports (162 a, 162 b, 162 c) to be connected with the connection pipes (164 a, 164 b, 164 c) of the collection bag 164 respectively. The collection bag 164 has a negative-pressure buffer zone 164 e and two fluid collection zones 164 f that communicate with each other. The negative-pressure buffer zone 164 e is located between the two fluid collection zones 164 f and isolated from the two fluid collection zones 164 f, i.e., fluids (e.g., air or liquids) cannot cross over a border 164 j between the negative-pressure buffer zone 164 e and the two fluid collection zones 164 f. The two fluid collection zones 164 f is connected with the first connection port 162 a of the multiple-pipe integration module 162 via the connection pipe 164 a. The negative-pressure buffer zone 164 e is connected with the second and third connection ports (162 b, 162 c) of the multiple-pipe integration module via the connection pipes (164 b, 164 c), and the negative-pressure buffer zone is equipped with a fluid input port 164 d. The fluid input port 164 d and the connection pipes (164 b, 164 c) are located at two opposite sides of the negative-pressure buffer zone 164 e. The negative-pressure buffer zone 164 e has a negative-pressure detection isolation section 164 e′ inside thereof to be connected with the third connection port 162 c of the multiple-pipe integration module 162. The negative-pressure detection isolation section 164 e′ accommodates a negative-pressure detection head assembly so as to detect a negative-pressure status of the negative-pressure buffer zone 164 e.

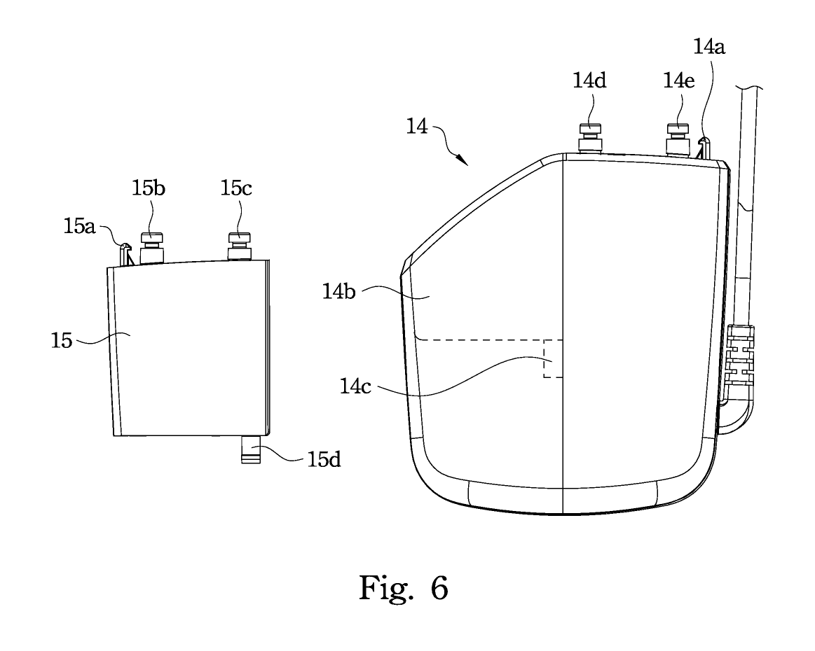

FIG. 6 illustrates an enlarged view of a vacuum driving unit and an actuator in FIG. 2. The actuator 14 has a concave trough 14 b to accommodate the vacuum driving unit 15, and the concave trough 14 b has a fastening slot 14 c. The vacuum driving unit 15 has a fastening hook 15 d to detachably engage the fastening slot 14 c of the actuator 14 such that the vacuum driving unit 15 can be secured within the concave trough 14 b of the actuator 14. The vacuum generator 15 has two connection ports (15 b, 15 c). The actuator 14 has a connection port 14 d of a negative pressure detector and a connection port 14 e of a positive pressure detector.

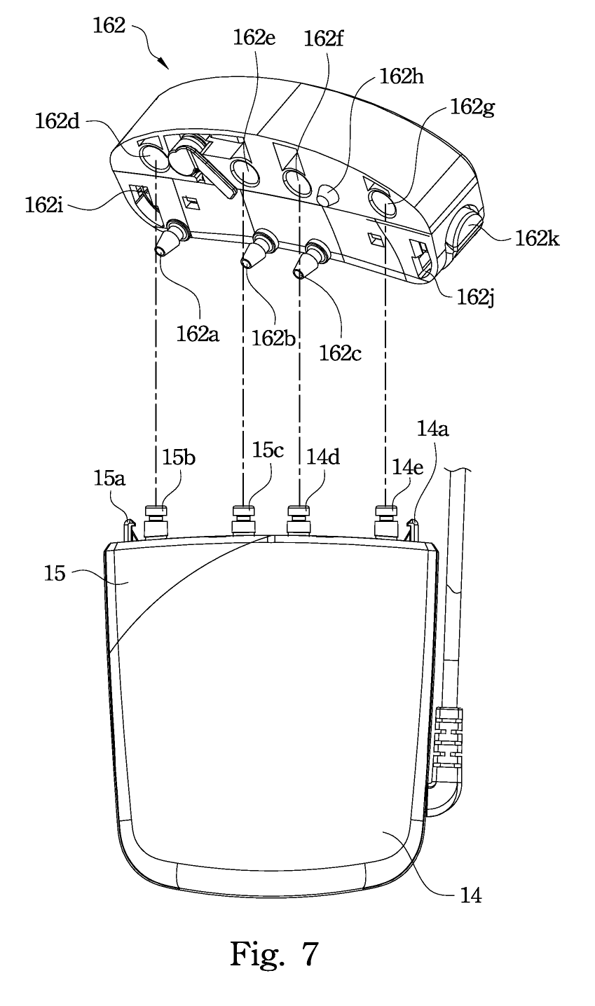

FIG. 7 illustrates a schematic view of the vacuum driving unit and the actuator connecting to the multiple-pipe integration module. After the vacuum driving unit 15 and the actuator 14 are assembled, the two connection ports (15 b, 15 c) of the vacuum generator, the connection port 14 d of the negative pressure detector and the connection port 14 e of the positive pressure detector are located on a common flat surface of a combined assembly of the vacuum driving unit 15 and the actuator 14. The first, second, third, fourth, fifth, sixth and seventh connection ports (162 a, 162 b, 162 c, 162 d, 162 e, 162 f, 162 g) are located on a common flat surface of the multiple-pipe integration module 162. The flat surface of the multiple-pipe integration module 162 on which the first, second, third, fourth, fifth, sixth and seventh connection ports (162 a, 162 b, 162 c, 162 d, 162 e, 162 f, 162 g) are located has two fastening slots (162 i, 162 j). The flat surface of the combined assembly of the vacuum driving unit 15 and the actuator 14 has two fastening hooks (14 a, 15 a) that detachably engage with the two fastening slots (162 i, 162 j) respectively so as to secure the multiple-pipe integration module 162 to the combined assembly of the vacuum driving unit 15 and the actuator 14.

When the multiple-pipe integration module 162 is fastened to the combined assembly of the vacuum driving unit 15 and the actuator 14, the two connection ports (15 b, 15 c) of the vacuum generator are detachably connected with the fourth and fifth connection ports (162 d, 162 e) of the multiple-pipe integration module 162 respectively, the connection port 14 d of the negative pressure detector is detachably connected with the sixth connection port 162 f of the multiple-pipe integration module 162, and the connection port 14 e of the positive pressure detector is detachably connected with the seventh connection port 162 g of the multiple-pipe integration module 162.

Referring to FIG. 5 and FIG. 7, when the connection port 14 d of the negative pressure detector is connected with the sixth connection port 162 f of the multiple-pipe integration module 162, the negative pressure detector can detect a negative-pressure status of the negative-pressure buffer zone 164 e because the third and sixth connection ports (162 c, 162 f) communicate with each other.

Referring to FIG. 5 and FIG. 7, when the connection port 14 e of the positive pressure detector is connected with the seventh connection port 162 g of the multiple-pipe integration module 16, the positive pressure detector can detect a positive-pressure status of the two fluid collection zones 164 f because the first, fourth and seventh connection ports (162 a, 162 d, 162 g) communicate with one another. Because the seventh connection port 162 g contains a backwater gate 162 n (see FIG. 4), the reverse fluid flows, e.g. tissue fluids, from the first or fourth connection ports (162 a, 162 d) will not pass through the backwater gate 162 n and damage the positive pressure detector.

When the multiple-pipe integration module 162 is desired to be detached from the combined assembly of the vacuum driving unit 15 and the actuator 14, a button 162 k is pressed to disengage the two fastening hooks (14 a, 15 a) from the two fastening slots (162 i, 162 j) of the multiple-pipe integration module 162. After the multiple-pipe integration module 162 is detached from the combined assembly of the vacuum driving unit 15 and the actuator 14, a push member 162 h does not contact a trigger member of the actuator 14 so as to stop a motor or detectors of the actuator 14 from operating.

Referring to both FIG. 5 and FIG. 7, when the vacuum driving unit 15 is operating to generate a negative pressure, the fluids within the negative-pressure buffer zone 164 e is drained via the connection pipe 164 b and directed into the two fluid collection zones 164 f via the connection pipe 164 a such that the negative-pressure level of the negative-pressure buffer zone 164 e is increased so as to suck more fluids via the fluid input port 164 d. When the fluids within the negative-pressure buffer zone 164 e is totally drained out, the vacuum driving unit 15 stops operating.

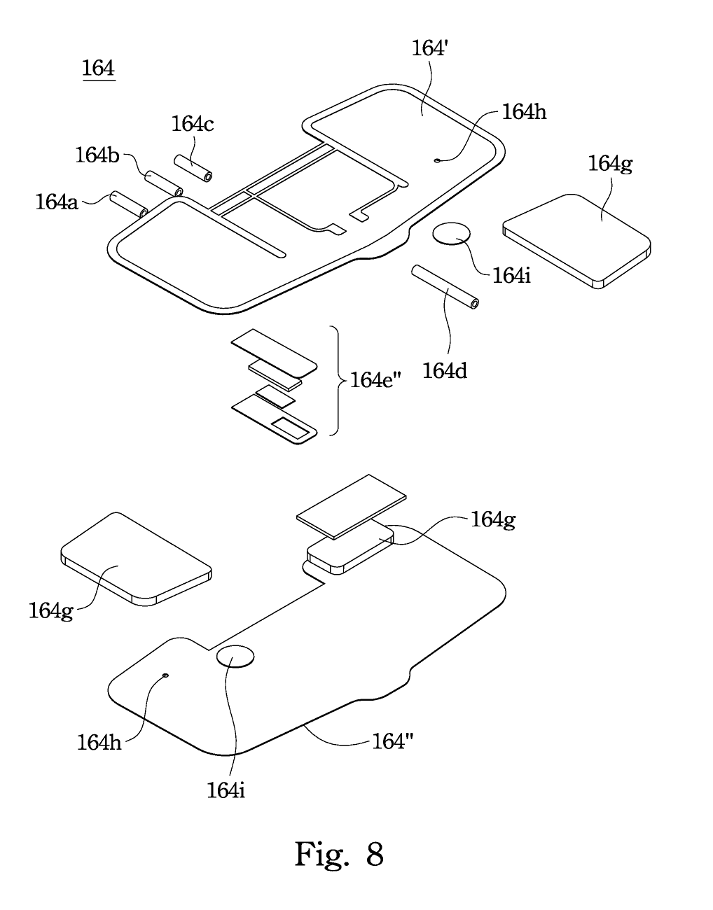

Referring to both FIG. 5 and FIG. 8, wherein FIG. 8 illustrates an exploded view of the collection bag in FIG. 5. The collection bag 164 basically includes an upper sheet 164′ and a lower sheet 164″ that are attached to sandwich the remaining components illustrated in the FIG. 8. The negative-pressure buffer zone 164 e and two fluid collection zones 164 f all have polyvinyl alcohol sheets 164 g inside thereof. Each polyvinyl alcohol sheet 164 g is used to absorb the fluids within the negative-pressure buffer zone or the fluid collection zone and thus maintains a workable air ventilation path. Each of the two fluid collection zones 164 f has a ventilation hole 164 h and a waterproof ventilation sheet 164 i that is attached to the ventilation hole 164 h such that excessive air within the two fluid collection zones 164 f can be exhausted out through the ventilation hole 164 h. The negative-pressure detection isolation section 164 e′ accommodates a negative-pressure detection head assembly 164 e″ so as to detect a negative-pressure status of the negative-pressure buffer zone 164 e.

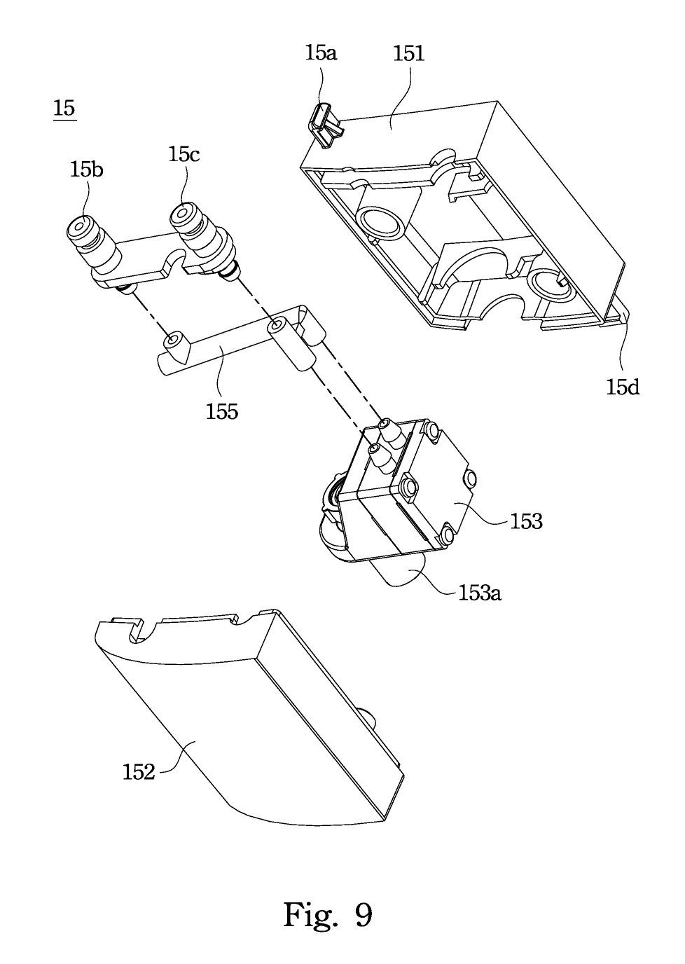

FIG. 9 illustrates an exploded view of the vacuum driving unit in FIG. 6. The vacuum driving unit 15 contains a vacuum generator 153 and two half housings (151, 152). The two half housings (151, 152) are assembled to enclose the vacuum generator 153. The vacuum generator 153 has a rotation shaft 153 a that is detachably connected with the motor of the actuator 14 such that the vacuum generator 153 can be driven to generate a negative-pressure. The vacuum generator 153 is connected with the upper two connection ports (15 b, 15 c) via the connection pipe 155.

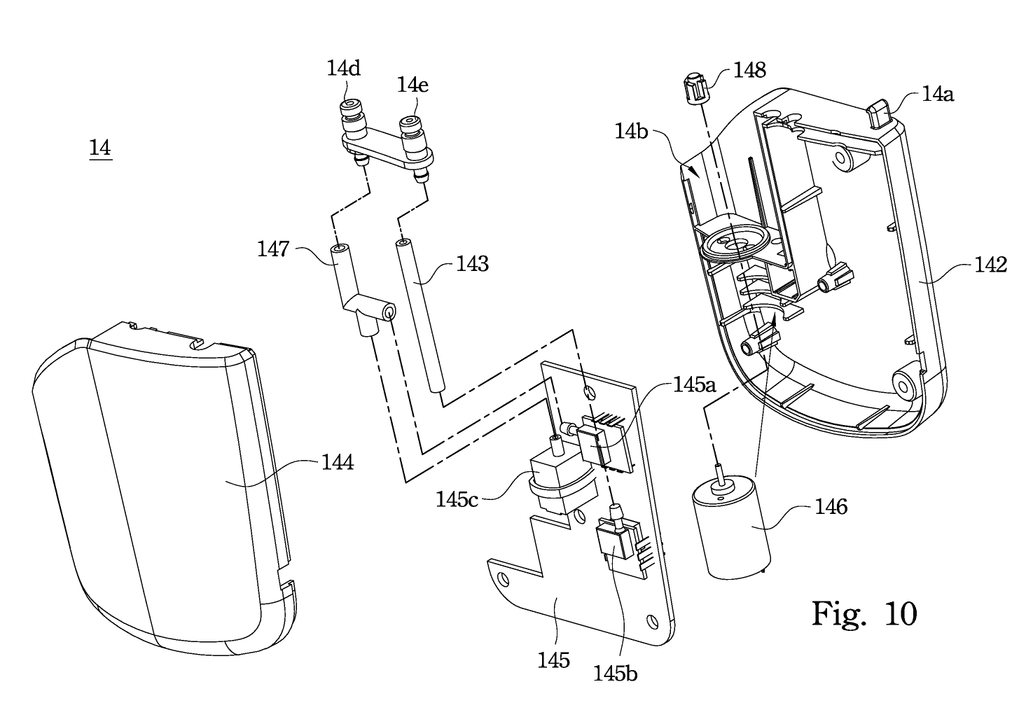

FIG. 10 illustrates an exploded view of the actuator in FIG. 6. The actuator 14 contains a motor 146, a negative pressure detector 145 a and a positive pressure detector 145 b. The actuator 14 has two half housings (142, 144). When the two half housings (142, 144) are assembled, the motor, the negative pressure detector and positive pressure detector are sandwiched therebetween. The motor 146 is fastened to the place to which an arrow is directed and its rotation shaft is connected to a shaft gear 148. When the vacuum driving unit 15 is assembled within the concave trough 14 b of the actuator 14, the shaft gear 148 is used to drive the vacuum generator 153 to generate a negative pressure. The negative pressure detector 145 a and positive pressure detector 145 b are both mounted on a circuit board 145. A three-way connection pipe 147 has an upper port connected to the connection port 14 d and two lower ports connected to the negative pressure detector 145 a and a pressure relief valve 145 c. A connection pipe 143 has an upper port connected to the connection port 14 e and a lower port connected to the positive pressure detector 145 b.

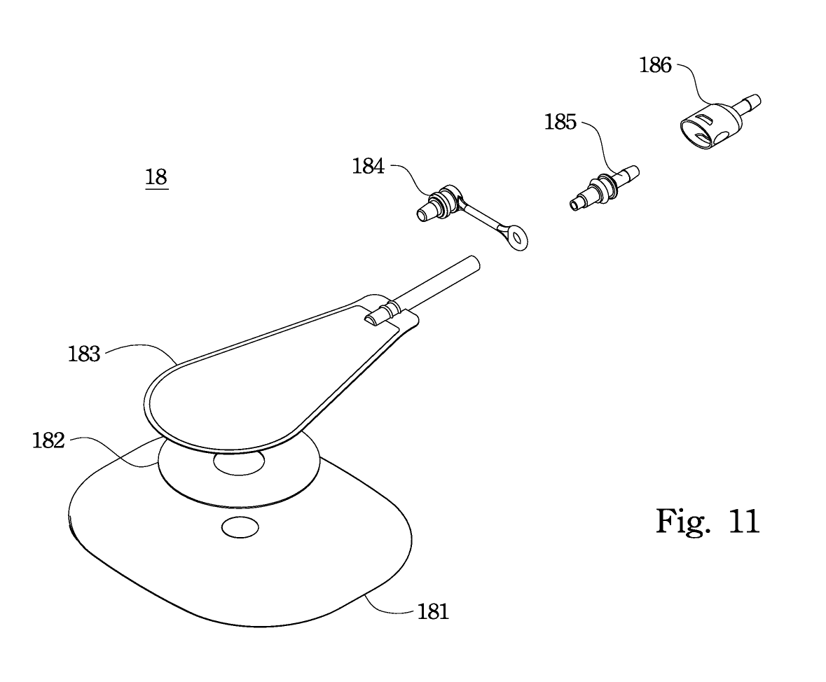

FIG. 11 illustrates an exploded view of a wound seal unit in FIG. 2. The wound seal unit 18 is attached to a wound such that the wound drainage therapy system can suck the tissue fluid of the wound through the wound seal unit 18. The wound seal unit 18 includes a tape 181, a filter sheet 182 and a bag 183 that are laminated together in accordance with the relative position illustrated in FIG. 11. The tape 181 is affixed to the skin around the wound such that the wound seal unit 18 can be attached to the wound. The wound seal unit 18 further has a male connecter 185, a female connecter 186 and a connecter hat 184 a. The male connecter 185 and the female connecter 186 can be detachably connected with each other. After the male connecter 185 is detached from the female connecter 186, the connecter hat 184 can be used to shield the male connector 185 to prevent the external contamination.

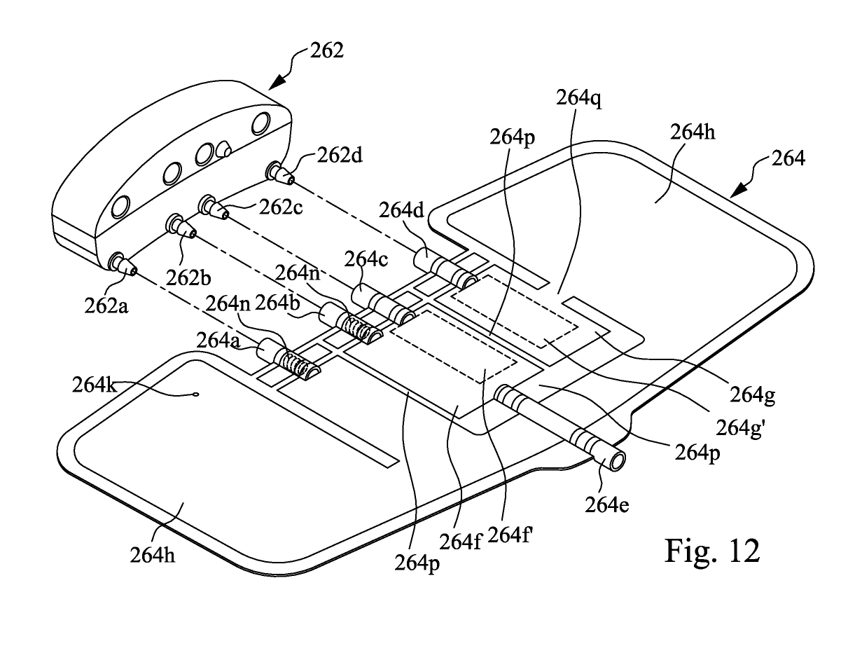

FIG. 12 illustrates an enlarged view of another embodiment of the multiple-pipe integration module and the collection container of the fluid collector unit. The fluid collector unit 26 includes a multiple-pipe integration module 262 and a collection container 264 (e.g., a collection bag or other likes). The multiple-pipe integration module 262 is a single body for integrating all tubes connected to the collection container 264. The collection container 264 has four connection pipes (264 a, 264 b, 264 c, 264 d) that are connected to the connection ports of the multiple-pipe integration module 262 respectively.

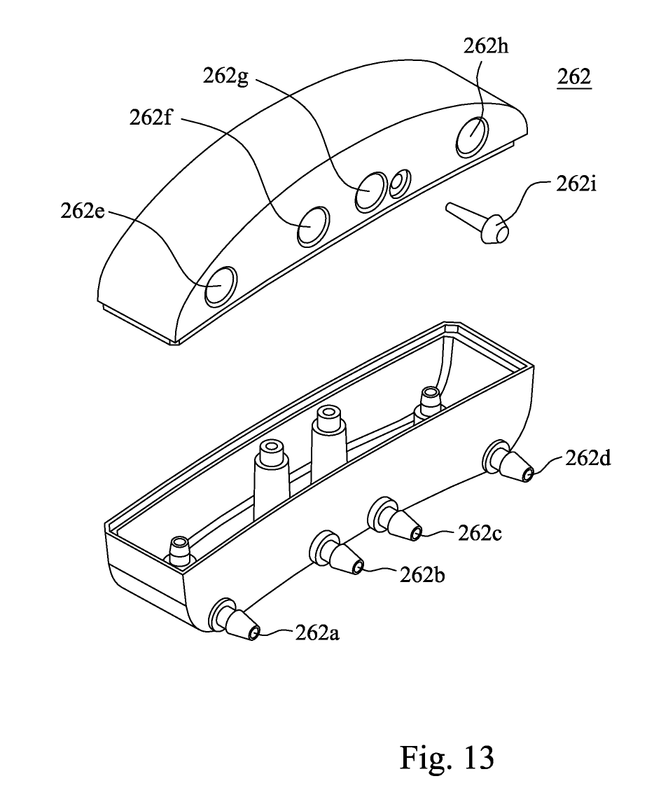

FIG. 13 illustrates an exploded view of the multiple-pipe integration module in FIG. 12. The multiple-pipe integration module 262 includes an upper half housing and a lower half housing to assemble a complete one. The multiple-pipe integration module 262 includes a first row connection port group and a second row connection port group. The first row connection port group includes first, second, third and fourth connection ports (262 a, 262 b, 262 c, 262 d), and the second row connection port group includes fifth, sixth, seventh and eighth connection ports (262 e, 262 f, 262 g, 262 h). The first and fifth connection ports (262 a, 262 e) communicate with each other, the second and sixth connection ports (262 b, 262 f) communicate with each other, the third and seventh connection ports (262 c, 262 g) communicate with each other, and the fourth and eighth connection ports (262 d, 262 h) communicate with each other.

Referring to FIG. 12, the first, second, third and fourth connection ports (262 a, 262 b, 162 c, 262 d) of the multiple-pipe integration module 262 are connected with the connection pipes (264 a, 264 b, 264 c, 264 d) of the collection container 264 respectively. In this embodiment, the connection pipe 264 a is a fluid input pipe, the connection pipe 264 b is a fluid output pipe, the connection pipe 264 c is a first gas output pipe, and the connection pipe 264 d is a second gas output pipe.

The collection container 264 has a negative-pressure buffer zone 264 f, a positive-pressure detection zone 264 g and a fluid collection zone 264 h. One side of the fluid collection zone 264 h communicates with the other side of the fluid collection zone 264 h. The negative-pressure buffer zone 264 f is isolated from the positive-pressure detection zone 264 g and the fluid collection zone 264 h, i.e., fluids (e.g., air or liquids) cannot cross over a border 264 p between the negative-pressure buffer zone 264 f and the positive-pressure detection zone 264 g or between the negative-pressure buffer zone 264 f and the fluid collection zone 264 h.

The fluid collection zone 264 h is connected with the first connection port 262 a of the multiple-pipe integration module 262 via the connection pipe 264 a. The negative-pressure buffer zone 264 f is connected with the second and third connection ports (262 b, 262 c) of the multiple-pipe integration module 262 via the connection pipes (264 b, 264 c), and the negative-pressure buffer zone 264 f is equipped with a fluid input port 264 e. The fluid input port 264 e and the connection pipes (264 b, 264 c) are located at two opposite sides of the negative-pressure buffer zone 264 f. The negative-pressure buffer zone 264 f has a negative-pressure detection isolation section 264 f′ inside thereof to be connected with the third connection port 262 c of the multiple-pipe integration module 262. The negative-pressure detection isolation section 264 f′ accommodates a negative-pressure detection head assembly so as to detect a negative-pressure status of the negative-pressure buffer zone 264 f.

The positive-pressure detection zone 264 g is connected with the fourth connection ports 262 d of the multiple-pipe integration module 262 via the connection pipe 264 d, and one side of the positive-pressure detection zone 264 g communicate with the fluid collection zone 264 h via a passage 264q. The positive-pressure detection zone 264 g has a positive-pressure detection isolation section 264 g′ inside thereof to be connected with the fourth connection port 262 d of the multiple-pipe integration module 262. The positive-pressure detection isolation section 264 g′ accommodates a positive-pressure detection head assembly so as to detect a positive-pressure status of the positive-pressure detection zone 264 g.

Referring to FIG. 6 and FIG. 13, the first, second, third, fourth, fifth, sixth, seventh and eighth connection ports (262 a, 262 b, 262 c, 262 d, 262 e, 262 f, 262 g, 262 h) are located on a common flat surface of the multiple-pipe integration module 262. When the multiple-pipe integration module 262 is fastened to the combined assembly of the vacuum driving unit 15 and the actuator 14, the two connection ports (15 b, 15 c) of the vacuum generator are detachably connected with the fifth and sixth connection ports (262 e, 262 f) of the multiple-pipe integration module 262 respectively, the connection port 14 d of the negative pressure detector is detachably connected with the seventh connection port 262 g of the multiple-pipe integration module 262, and the connection port 14 e of the positive pressure detector is detachably connected with the eighth connection port 262 h of the multiple-pipe integration module 262.

After the multiple-pipe integration module 262 is detached from the combined assembly of the vacuum driving unit 15 and the actuator 14, a push member 262 i does not contact a trigger member of the actuator 14 so as to stop a motor or detectors of the actuator 14 from operating.

Referring to FIG. 6 and FIG. 12, when the connection port 14 d of the negative pressure detector is connected with the seventh connection port 262 g of the multiple-pipe integration module 262, the negative pressure detector can detect a negative-pressure status of the negative-pressure buffer zone 264 f because the third and seventh connection ports (262 c, 262 g) communicate with each other.

Referring to FIG. 6 and FIG. 12, when the connection port 14 e of the positive pressure detector is connected with the eighth connection port 262 h of the multiple-pipe integration module 262, the positive pressure detector can detect a positive-pressure status of the fluid collection zone 264 h because the fourth and eighth connection ports (262 d, 262 h) communicate with each other.

Referring to both FIG. 6 and FIG. 12, when the vacuum driving unit 15 is operating to generate a negative pressure, the fluids within the negative-pressure buffer zone 264 f is drained via the connection pipe 264 b and directed into the fluid collection zone 264 h via the connection pipe 264 a such that the negative-pressure level of the negative-pressure buffer zone 264 f is increased so as to suck more fluids via the fluid input port 264 e. Furthermore, air within the negative-pressure buffer zone 264 f can be outputted via the connection pipe 264 c and detected by the negative pressure detector to obtain the negative-pressure status of the negative-pressure buffer zone 264 f; similarly, air within the fluid collection zone 264 h can be outputted via the connection pipe 264 d and detected by the positive pressure detector to obtain the positive-pressure status of the fluid collection zone 264 h.

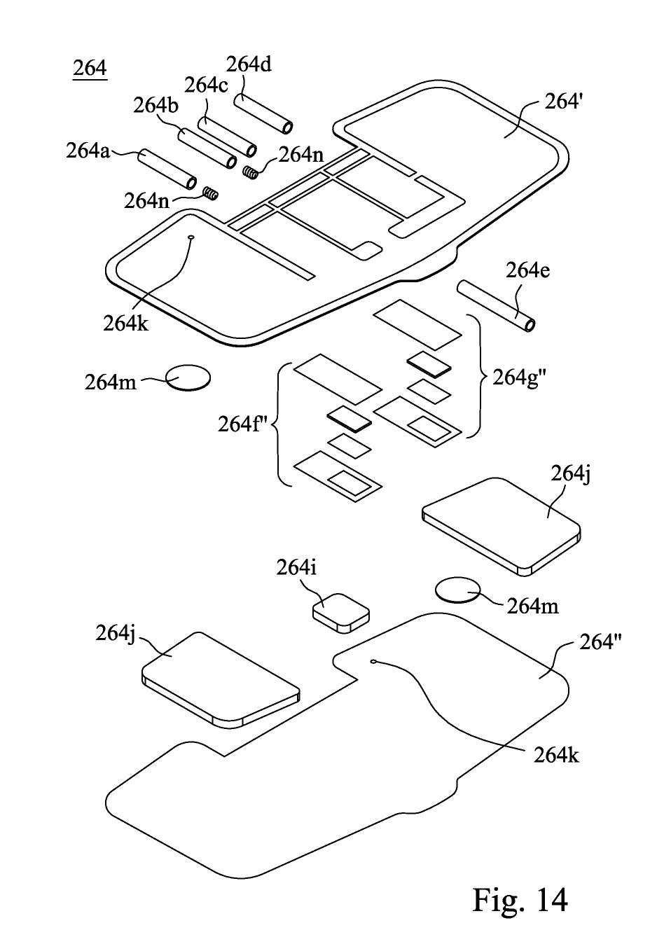

Referring to both FIG. 12 and FIG. 14, wherein FIG. 14 illustrates an exploded view of the collection container in FIG. 12. The collection container 264 basically includes an upper sheet 264′ and a lower sheet 264″ that are attached to sandwich the remaining components illustrated in the FIG. 14. In this embodiment, the negative-pressure buffer zone 264 f has a polyvinyl alcohol sheet 264 i inside thereof and the fluid collection zone 264 h has superabsorbent polymer materials 264 j, such as polyvinyl alcohol sheets or sodium polyacrylate sheets inside thereof. The polyvinyl alcohol sheet 264 i is used to absorb and exhaust the fluids within the negative-pressure buffer zone 264 f quickly and thus perform temporary storage of pressure. The superabsorbent polymer materials 264 j are used to absorb the fluids within the fluid collection zone 264 h effectively.

Furthermore, various additives can be added into the superabsorbent polymer materials 264 j. For example, deodorant additives, such as sodium bicarbonate, activated carbon or chitin, are used to reduce the smell of the fluids; antibacterial additives, such as nano-silver, titanium dioxide or zinc oxide, are used to inhibit the growth of the bacteria in the container.

The fluid collection zone 264 h has at least one ventilation hole 264 k and at least one waterproof ventilation sheet 264 m that is attached to the at least one ventilation hole 264 k such that excessive air within the fluid collection zone 264 h can be exhausted out through the at least one ventilation hole 264 k.

The negative-pressure detection isolation section 264 f′ accommodates a negative-pressure detection head assembly 264 f″ so as to detect a negative-pressure status of the negative-pressure buffer zone 264 f. Similarly, the positive-pressure detection isolation section 264 g′ accommodates a positive-pressure detection head assembly 264 g″ so as to detect a positive-pressure status of the positive-pressure detection zone 264 g.

Furthermore, each of the connection pipe 264 a and the connection pipe 264 b has a muffler element inside thereof. The muffler element is used to decrease vibrations occurred by the straight flow of the fluids. The muffler element can be a muffler screw which can form a spiral flow or other likes.

According to the above-discussed embodiments, the wound drainage therapy system disclosed herein has modularized parts which can be easily assembled and disassembled with each other, the multiple-pipe integration module of which overcomes the piping entanglement problem, and the fluid collector unit is designed smaller to enable the wound drainage therapy system even more portable.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A fluid collector unit of a wound drainage therapy system comprising:
a multiple-pipe integration module comprising first, second and third connection ports; and
a collection bag comprising a negative-pressure buffer zone and two fluid collection zones that communicate with each other, the negative-pressure buffer zone being located between the two fluid collection zones and isolated from the two fluid collection zones, the two fluid collection zones being connected with the first connection port of the multiple-pipe integration module, the negative-pressure buffer zone being connected with the second and third connection ports of the multiple-pipe integration module, the negative-pressure buffer zone having a fluid input port.

2. The fluid collector unit of claim 1, wherein the negative-pressure buffer zone has a negative-pressure detection isolation section that is connected with the third connection port of the multiple-pipe integration module.

3. The fluid collector unit of claim 1, wherein the negative-pressure buffer zone and the two fluid collection zones all have polyvinyl alcohol sheets inside thereof.

4. The fluid collector unit of claim 1, wherein the multiple-pipe integration module further comprises fourth, fifth, sixth and seventh connection ports, and the first, fourth and seventh connection ports communicate with one another, the second and fifth connection ports communicate with each other, the third and sixth connection ports communicate with each other, the seventh connection port contains a backwater gate inside to stop flows reversed from the first or fourth connection ports.

5. The fluid collector unit of claim 4, wherein the first, second, third, fourth, fifth, sixth and seventh connection ports are located on a common flat surface of the multiple-pipe integration module.

6. The fluid collector unit of claim 5, wherein each of the two fluid collection zones has a ventilation hole and a waterproof ventilation sheet that is attached to the ventilation hole.

7. A fluid collector unit of a wound drainage therapy system comprising:
a multiple-pipe integration module comprising first, second, third and fourth connection ports; and
a collection container comprising a negative-pressure buffer zone, a positive-pressure detection zone and a fluid collection zone, the negative-pressure buffer zone being isolated from the positive-pressure detection zone and the fluid collection zone, the fluid collection zone being connected with the first connection port of the multiple-pipe integration module, the positive-pressure detection zone being connected with the fourth connection port of the multiple-pipe integration module and communicating with the fluid collection zone, the negative-pressure buffer zone being connected with the second and third connection ports of the multiple-pipe integration module, the negative-pressure buffer zone having a fluid input port.

8. The fluid collector unit of claim 7, wherein the negative-pressure buffer zone has a negative-pressure detection isolation section that is connected with the third connection port of the multiple-pipe integration module; and the positive-pressure detection zone has a positive-pressure detection isolation section that is connected with the fourth connection port of the multiple-pipe integration module.

9. The fluid collector unit of claim 7, wherein the negative-pressure buffer zone has a polyvinyl alcohol sheet inside thereof, and the fluid collection zone has superabsorbent polymer materials inside thereof.

10. The fluid collector unit of claim 7, wherein the multiple-pipe integration module further comprises fifth, sixth, seventh and eighth connection ports, the first and fifth connection ports communicate with each other, the second and sixth connection ports communicate with each other, the third and seventh connection ports communicate with each other, and the fourth and eighth connection ports communicate with each other.

11. The fluid collector unit of claim 10, wherein the first, second, third, fourth, fifth, sixth, seventh and eighth connection ports are located on a common flat surface of the multiple-pipe integration module.

12. The fluid collector unit of claim 7, wherein the fluid collection zone has at least one ventilation hole and at least one waterproof ventilation sheet that is attached to the at least one ventilation hole.

13. The fluid collector unit of claim 7, further comprising a first connection pipe and a second connection pipe, the first connection pipe connecting the fluid collection zone with the first connection port of the multiple-pipe integration module, the second connection pipe connecting the negative-pressure buffer zone with the second connection port of the multiple-pipe integration module, wherein each of the first connection pipe and the second connection pipe has a muffler element inside thereof.

14. A collection container comprising:
a negative-pressure portion comprising a negative-pressure buffer zone, a fluid input port, a fluid output pipe and a first gas output pipe, the fluid output pipe, each of the first fluid input pipe and the first gas output pipe being communicated with the negative-pressure buffer zone; and
a positive-pressure portion comprising a positive-pressure detection zone, a fluid collection zone, a second fluid input pipe and a second gas output pipe, the negative-pressure buffer zone being isolated from the fluid collection zone and the positive-pressure buffer zone, the second fluid input pipe being communicated with the fluid collection zone, the second gas output pipe being communicated with the positive-pressure detection zone, the positive-pressure detection zone being communicated with the fluid collection zone.

15. The collection container of claim 14, wherein the negative-pressure buffer zone has a negative-pressure detection isolation section that is connected with the first gas output port; and the positive-pressure detection zone has a positive-pressure detection isolation section that is connected with the second gas output port.

16. The collection container of claim 14, wherein the negative-pressure buffer zone has a polyvinyl alcohol sheet inside thereof, and the fluid collection zone has superabsorbent polymer materials inside thereof.

17. The collection container of claim 16, wherein the superabsorbent polymer materials are polyvinyl alcohol sheets or sodium polyacrylate sheets.

18. The collection container of claim 16, wherein the superabsorbent polymer materials have deodorant additives or antibacterial additives inside thereof.

19. The collection container of claim 14, wherein the fluid collection zone has at least one ventilation hole and at least one waterproof ventilation sheet that is attached to the at least one ventilation hole.

20. The collection container of claim 14, wherein each of the first fluid output pipe and the second fluid input pipe has a muffler element inside thereof.
